(19) United States
(12) Patent Application Publication
(10) Pub. No.: US 2009/0228040 A1
(43) Pub. Date: Sep. 10, 2009

(54) Mechanism and Method for Closing an Arteriotomy

(75) Inventors: Juan-Pablo Mas, Somerville, MA (US); David Yann, Lynn, MA (US); Matthew Spurchise, Peabody, MA (US); Thierry Benjamin, Lowell, MA (US); Richard Rego, Mansfield, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/041,777

(22) Filed: Mar. 4, 2008

(51) Int. Cl. A61B 17/08; A61B 17/064

(52) U.S. Cl. 606/216; 606/219

(57) ABSTRACT

Devices and methods for closing an arteriotomy include a percutaneously placeable central tube deployable about the arteriotomy by which an array of radially expandable fingers having suction ports can be engaged with the surface of tissue about the arteriotomy enabling the fingers to grip the tissue without piercing it. A delivery tube can advance a closure element along the outside surface of the central tube, thereby radially contracting the fingers and puckering the gripped tissue to approximate the edges of the arteriotomy. The closure element is further advanced until it is forced off of the distal tips of the fingers such that the closure element surrounds the puckered tissue and secures permanent closure of the arteriotomy.

### FIELD OF THE INVENTION

The invention relates in general to devices and techniques for closing a percutaneous puncture in a blood vessel after an intravascular procedure.

### BACKGROUND OF THE INVENTION

Various cardiovascular procedures, such as angioplasty, stent placement and atherectomy, require inserting into and manipulating within the vasculature, wires and catheters adapted to perform those procedures. Access to the vasculature typically is through the femoral artery and is percutaneous, involving insertion of a needle and introducer sheath in the region of the groin to form a track through subcutaneous tissue and to puncture and create an arteriotomy in the femoral artery. A short guidewire is then advanced through the needle and into the femoral artery. The needle then is removed. An introducer sheath is then advanced over the guidewire, along the track and into the femoral artery. The sheath provides access into the femoral artery, through the arteriotomy, for catheters or other instrumentalities in order to perform the selected procedure.

After the procedure has been completed, the procedural devices are removed and the arteriotomy must be closed. A number of techniques are known to facilitate closure and healing of the arteriotomy. These include application of pressure at the puncture site for a relatively extended length of time, or the use of biological adhesives or plugs adapted to seal the arteriotomy, or the use of staples or clips. Some closure systems include an arrangement to engage the artery to temporarily draw the edges of the arteriotomy together while a final closure element, such as a staple, sutures, adhesives or other means is used to affect the permanent closure of the arteriotomy. Such systems are described, for example, in U.S. Pat. No. 6,767,356 (Kanner) and U.S. Pat. No. 6,391,048 (Ginn et al.). Ginn discloses an arrangement in which several needles pierce the vessel wall surrounding the arteriotomy and then are manipulated to twist or draw together the vessel wall about the arteriotomy. Adhesives, sutures or clips then may be employed to secure a permanent closure. However, it would be desirable to provide a closure system in which tissue about the arteriotomy could be temporarily drawn together without risking the trauma from piercing the tissue. Accordingly, the present invention is directed to such an alternate mechanism and technique for closing an arteriotomy.

In addition, it is desirable to provide such a closure system with a very low profile. Some final closure elements, including staples, sutures, adhesives or other means used to affect the permanent closure of the arteriotomy, require relatively large delivery devices that actually enlarge the size of the arteriotomy when delivering the closure element to the target tissue. Accordingly, it is an object of the present invention to provide a closure element delivery device having a low profile in order to prevent incidental enlargement of the arteriotomy.

### BRIEF SUMMARY OF THE INVENTION

Embodiments of the present invention relate to a system for closing an arteriotomy. The system includes an elongate central tube having a distal portion including a plurality of gripping elements disposed thereon, the plurality of gripping elements configured to be movable between a radially contracted configuration and a radially expanded configuration. The gripping elements may be actuated to effect a grip on tissue surrounding the arteriotomy when the gripping elements are in the radially expanded configuration. The system also includes a delivery tube disposed on an outside surface of the central tube, the delivery tube being slidable along the central tube to urge the gripping elements from the radially expanded configuration to the radially contracted configuration while maintaining grip on the tissue whereby the tissue may be puckered together. A closure element is disposed on the outside surface of the central tube, the closure element being slidable along and off a distal end of the central tube by the delivery tube and adapted to surround the puckered tissue to close the arteriotomy.

Embodiments of the present invention also relate to a method for closing an arteriotomy. A plurality of tissue gripping elements on a distal portion of a central tube is provided, the gripping elements being configured to be selectively movable between a radially contracted configuration and a radially expanded configuration. The gripping elements are positioned in the radially expanded configuration and disposed in surrounding relation to the arteriotomy. The gripping elements are actuated to cause them to grip the tissue surface without piercing the tissue. A closure element is provided around the outside surface of the central tube, and a delivery tube is provided about the central tube such that the delivery tube can push the closure element along the outside surface of the central tube. The delivery tube and the closure element are slidably advanced distally over the outside of the central tube until the gripping elements are collapsed to the radially contracted configuration while maintaining grip on the tissue surface whereby the tissue surrounding the arteriotomy may be puckered together. The delivery tube and the closure element are further slidably advanced distally along the outside of the central tube until the closure element is pushed off of a distal end of the central tube by the delivery tube such that the closure element surrounds the puckered tissue and permanently secures the arteriotomy closed. Thereafter the gripping elements are released from the tissue.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a mechanism at the distal portion of the arteriotomy closure system of the present invention, as seen from a proximal oblique angle.

FIG. 2 illustrates the mechanism at the distal portion of the arteriotomy closure system shown in FIG. 1, as seen from a distal oblique angle.

FIG. 3 is a perspective illustration of the mechanism of FIGS. 1 and 2, with tissue-gripping retention fingers in a deployed position to be disposed against tissue about the arteriotomy.

FIG. 4 is a perspective illustration of the mechanism of FIGS. 1-3, with a closure element in position for advancement towards the deployed tissue-gripping retention fingers.

FIG. 5 illustrates the mechanism of FIGS. 1-4, with a delivery tube pushing the closure element along the outer surface of the tissue-gripping retention fingers, thereby radially contracting the fingers.

FIG. 6 illustrates the mechanism of FIGS. 1-5, with the closure element being pushed off of the distal tips of the contracted tissue-gripping retention fingers.

FIG. 7 is a transverse cross-sectional illustration of a mechanism at the distal portion of an arteriotomy closure system in accordance with the present invention, with the fingers radially contracted to purse the tissue about an arteriotomy, and with the guidewire having been removed for clarity.

FIG. 8 is a perspective illustration of another embodiment of a mechanism at the distal portion of an arteriotomy closure system in accordance with the present invention, as seen from a proximal oblique angle, wherein the system includes a protective sheath.

FIG. 9 is a perspective illustration of a delivery tube of an arteriotomy closure system according to another embodiment of the present invention.

FIG. 10 is a partial longitudinal sectional view of an arteriotomy closure system of the present invention, wherein the system includes the delivery tube of FIG. 9.

FIG. 11 is an illustration of the closure element according to one embodiment of the present invention, wherein the closure element is an annular band.

FIG. 12 is an illustration of the closure element according to another embodiment of the present invention, wherein the closure element is a U-shaped gripping clip.

FIG. 13A is a top view of the closure element according to another embodiment of the present invention, wherein the closure element is a spiral clip.

FIG. 13B is a perspective view of the spiral clip closure element illustrated in FIG. 13A.

FIG. 14 is a perspective illustration of the closure element according to another embodiment of the present invention, wherein the closure element is a plug.

FIG. 15 is a perspective illustration of the closure element according to another embodiment of the present invention, wherein the closure element is a pronged staple.

FIG. 16 is a side view of a central tube of the arteriotomy closure system according to another embodiment of the present invention, wherein the central tube includes a ridge at the distal end for engaging and closing a pronged staple.

FIGS. 17-18 are partial longitudinal sectional views that illustrate, diagrammatically, the manner in which the central tube of FIG. 16 may be used to engage and close a pronged staple.

FIG. 19 is a transverse cross-section illustrating the closure element disposed around the outer surface of the central tube according to one embodiment of the present invention.

FIG. 20 is a transverse cross-section illustrating the contracted tissue-gripping retention fingers having the closure element around the outer surface thereof according to another embodiment of the present invention.

FIG. 21 illustrates the distal portion of a modified central tube to form the tissue-gripping retention fingers according to one embodiment of the present invention.

FIG. 22 illustrates the distal portion of a modified central tube to form the tissue-gripping retention fingers according to another embodiment of the present invention.

FIG. 23 is a perspective illustration of an embodiment of the present invention including pull wires for expanding the tissue-gripping retention fingers.

FIG. 24 is a perspective illustration of another embodiment of the present invention including pull wires for expanding the tissue-gripping retention fingers.

FIG. 25 is a longitudinal sectional illustration of an arteriotomy closure system in accordance with the present invention, showing a control module at the proximal end for communicating suction to the distal end and for enabling advancement or withdrawal of the delivery tube with respect to the central tube.

FIGS. 26-29 illustrate the manner in which an arteriotomy closure system of the present invention may be used to approximate the edges of an arteriotomy by engaging and drawing together connective tissue associated with the vessel in which the arteriotomy is formed.

FIG. 30 is a sectional illustration of a central tube in accordance with the present invention used in association with a stabilizing system in engagement with the vessel.

FIG. 31 is a perspective illustration of another embodiment of a stabilizing system as may be used in connection with the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Thus, the meaning of the term arteriotomy, as used for convenience throughout the specification and claims, should be taken to include openings in body passageways in addition to arteries, such as, by non-limiting example, a venipuncture into a vein. Furthermore, there is no intention to be bound by any expressed or implied theory presented in the preceding technical field, background, brief summary or the following detailed description.

Embodiments of the present invention relate to a device for delivering a closure element that closes an arterial puncture or arteriotomy. More particularly, a percutaneously placeable central tube is deployable about an arteriotomy by which an array of tissue grippers or fingers having suction ports can be engaged with the surface of tissue about the arteriotomy enabling the ports to grip the tissue without piercing it. The device may be advanced over an indwelling guidewire with the fingers in a radially contracted configuration. When the distal end of the device is located at the region of the arteriotomy, the fingers then may be deployed to a more radially expanded configuration about the region of the arteriotomy and, when in position, the gripping fingers may be actuated by applying suction to the suction ports to grip the tissue. With the fingers holding the tissue, the closure element can be distally advanced over the outer surface of the central tube to radially contract or collapse the fingers, thereby gathering, pursing, or puckering the tissue together to close the arteriotomy. The closure element is delivered to the site of the puckered arteriotomy by further distally advancing the closure element until it is released or forced over the distal tips of the fingers and surrounds the puckered tissue. The fingers then may be operated to release the tissue, as by terminating the suction, and the central tube and array of tissue fingers are withdrawn. The closure element remains in place to secure permanent closure of the arteriotomy. Since the closure element is advanced over the outer surface of the central tube, the central tube need only be of sufficient size to accommodate a conventional guidewire through a central lumen thereof and thus the overall profile of the delivery device can be minimized.

The closure element is advanced over the central tube by a delivery tube. In one embodiment, the delivery tube has a uniform or constant inner diameter along the length thereof. The inner diameter of the delivery tube is slightly larger than the outer diameter of the central tube in order to insure relative sliding motion between the delivery tube and the central tube. The closure element is pushed by the distal end of the delivery tube until it is released or forced off of the distal tips of the fingers and surrounds the puckered tissue.

In another embodiment, the delivery tube has a stepped inner diameter along the length thereof such that the distal portion of the delivery tube has a larger inner diameter than the proximal portion of the delivery tube. The closure element resides within the distal portion having a larger inner diameter such that the closure element is nested within the delivery tube during delivery. This “nested” embodiment ensures that the closure element is pushed over the fingers with minimal deformation and ensures that the closure element is protected during advancement. The closure element is pushed by the step of the inner diameter of the delivery tube until it is released or forced off of the distal tips of the fingers and surrounds the puckered tissue.

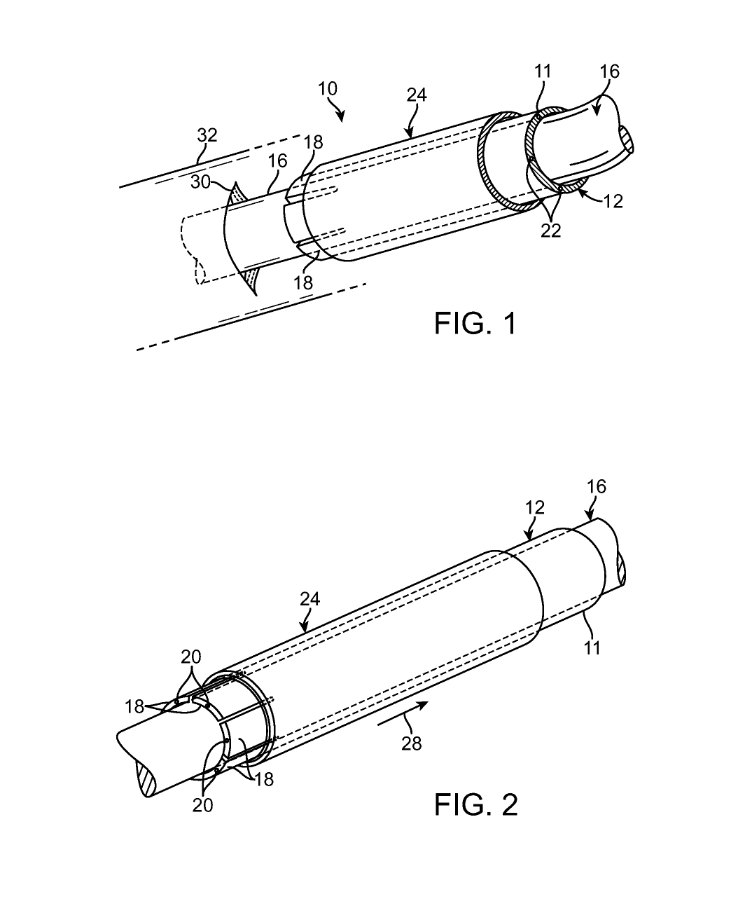

The closure element may take various forms, including an annular band, a spiral clip, a U-shaped clip, a plug with swelling potential, or a pronged staple. In one embodiment of the present invention, the closure element may be formed of a material having elastic properties in order to elastically grip the puckered tissue in order to secure permanent closure of the arteriotomy and to prevent slippage of the closure element. In another embodiment, the closure element may be formed from a material that plastically deforms in order to frictionally grip the puckered tissue about the arteriotomy and thereby prevent slippage of the closure element. Further details and description of the embodiments of the present invention are provided below with reference to FIGS. 1-31.

FIG. 1 illustrates the distal portion of a mechanism 10 incorporating the present invention, it being understood that the components of mechanism 10 that are movable along an axial direction (i.e., along a guidewire) extend to the proximal end of the system where they may be controlled to perform their respective functions and movements at the distal portion. Mechanism 10, in this embodiment, is a delivery system for delivering a closure device to the site of an arteriotomy 30 of an artery 32. Mechanism 10 includes a central tube 12 having a central lumen adapted to receive a guidewire 16. Since the closure element is slid along the outside of central tube 12, the central lumen therethrough need only be of sufficient size to accommodate guidewire 16, and thus the overall profile, e.g., diameter, of mechanism 10 is lower than it would be if the closure element were delivered through the central lumen. For example, a conventional medical guidewire typically has a diameter of between approximately 0.012 inches and 0.038 inches, and thus the central lumen of central tube 12 need be only slightly larger than the size of the intended guidewire in order to insure relative sliding motion between guidewire 16 and central tube 12. The distal portion of the central tube 12 carries radially expandable and contractible elements, such as tissue retention fingers 18 that may be formed integrally with central tube 12. Fingers 18 have gripping elements, such as suction ports 20 (shown in FIG. 2), at their distal ends. Suction ports 20 do not puncture tissue and are in communication with a source of suction (not shown) by suction lumens 22 extending proximally along central tube 12.

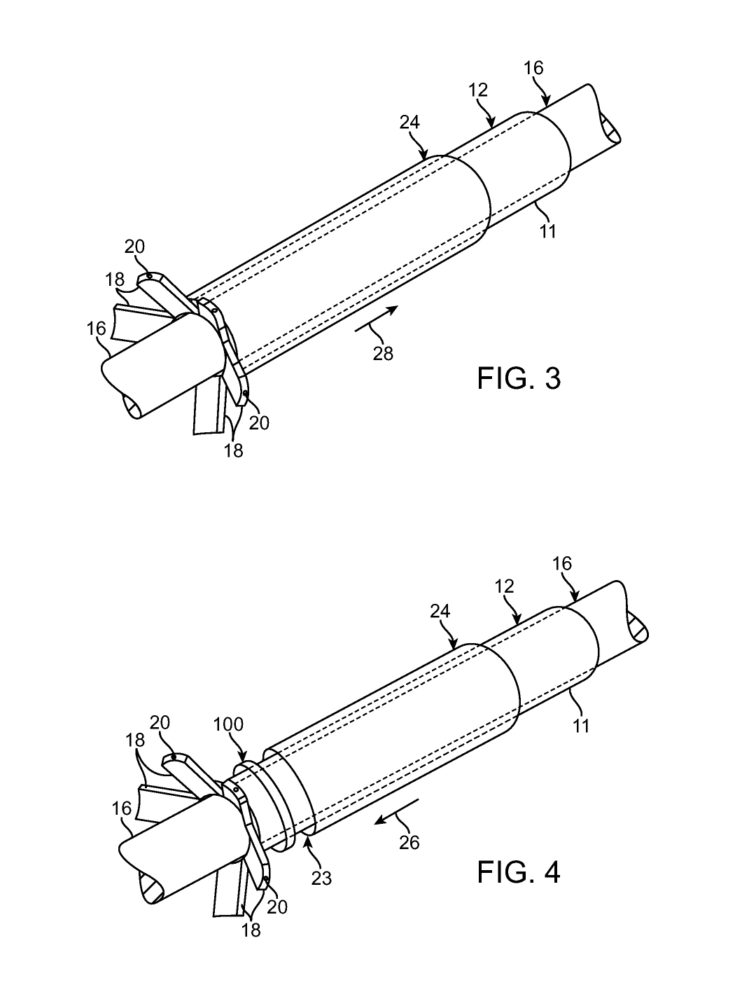

Fingers 18 are movable between a radially contracted, low profile configuration (illustrated in FIGS. 1, 2, 5, and 6) and a radially expanded, deployed configuration (illustrated in FIGS. 3 and 4) by a delivery tube 24 that is slidably disposed about an outer surface 11 of central tube 12. As will be explained in more detail here, fingers 18 may be caused to deploy to a radially expanded configuration by forming them with a resilient bias toward the expanded configuration or by an arrangement of pull wires, or a combination of both. Fingers 18 may be caused to flare outwardly to engage the surface of tissue about arteriotomy 30 and suction then can be applied to cause fingers 18 to grip the adjacent tissue surface. Fingers 18 then are drawn radially inwardly to pucker the tissue surrounding arteriotomy 30 and hold it in that configuration until a more permanent closure element is delivered over outer surface 11 of central tube 12.

More particularly, referring also to FIGS. 4-6, in addition to collapsing fingers 18 to a contracted configuration, delivery tube 24 also operates to push or force a closure element 100 over outer surface 11 of central tube 12 in order to deliver closure element 100 to the site of arteriotomy 30. Delivery tube 24 extends to the proximal end of the device and can be advanced distally (in the direction of arrow 26) such that a distal end 23 of delivery tube 24 distally advances closure element 100. Delivery tube 24 has a uniform or constant inner diameter along the length thereof. The inner diameter of delivery tube 24 is slightly larger than outer surface 11 of central tube 12 in order to permit relative sliding motion between delivery tube 24 and central tube 12. As closure element 100 and delivery tube 24 pass over fingers 18, fingers 18 radially contract or collapse, thereby puckering the tissue together and closing arteriotomy 30. In some embodiments of the present invention, closure element 100 is elastic or otherwise variable in its radial dimensions, as will be described below. In at least these embodiments, it is the radial stiffness of delivery tube 24 that causes fingers 18 to contract as tube distal end 23 passes thereover. Closure element 100 is further advanced by distal end 23 of delivery tube 24 until it is pushed or forced off of distal tips 19 of fingers 18 and surrounds the puckered tissue. Closure element 100 secures permanent closure of arteriotomy 30 when central tube 12 and fingers 18 are withdrawn.

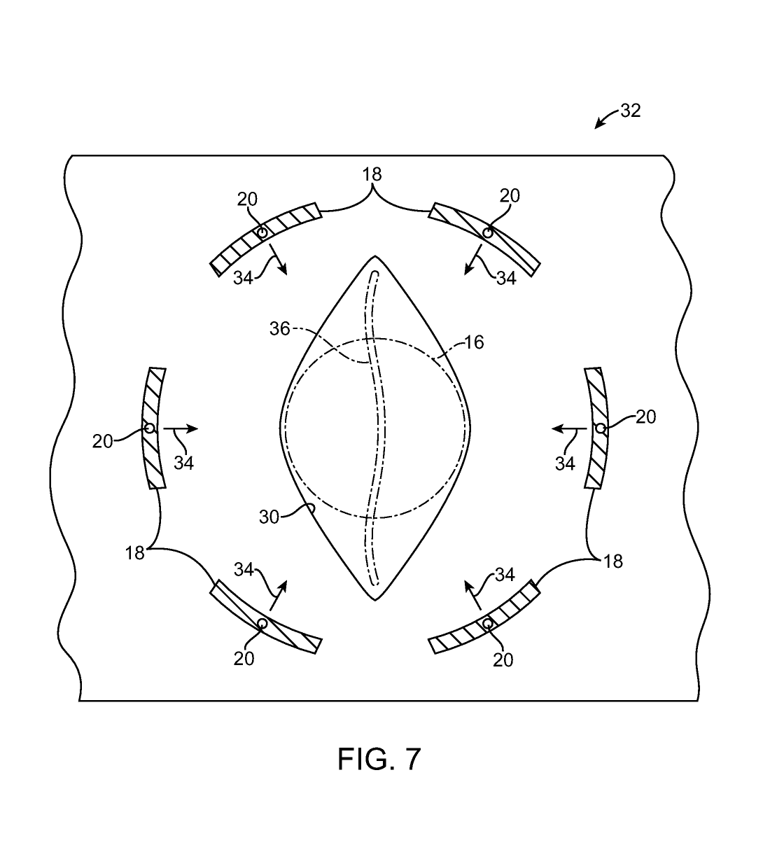

FIGS. 1-7 illustrate the manner or process in which mechanism 10 may be used to close an arteriotomy 30. While arteriotomy 30 and artery 32 are illustrated in FIG. 1, they have been removed from FIGS. 2-6 for clarity purposes. However, the description for utilizing mechanism 10 will describe the process for utilizing mechanism 10 in relation to arteriotomy 30. Referring to FIG. 1, central tube 12 is advanced along an indwelling guidewire 16 with fingers 18 maintained in a contracted configuration to facilitate delivery of the device through a tissue track. In order to maintain fingers 18 in the contracted position, delivery tube 24 surrounds fingers 18, and both the delivery tube and central tube 12 are advanced to the region of arteriotomy 30. Once the distal end of the device is in proximity to the region of arteriotomy 30, such as the tissue of the femoral sheath that surrounds artery 32, delivery tube 24 may be withdrawn proximally (in the direction of arrow 28) as shown in FIG. 2. Proximally withdrawing delivery tube 24 allows fingers 18 to deploy to a radially expanded configuration as illustrated in FIGS. 3 and 7.

In one embodiment, delivery tube 24 is withdrawn proximally until the distal end 23 of the delivery tube 24 is released from central tube 12 and/or guidewire 16. Once delivery tube 24 is removed, closure element 100 is positioned over outer surface 11 of central tube 12. In addition, delivery tube 24 is also positioned over outer surface 11 of central tube 12 such that distal end 23 of delivery tube 24 is proximal to closure element 100. Now referring to FIG. 4, delivery tube 24 is in position to be advanced distally (in the direction of arrow 26) in order to push closure element 100 distally along outer surface 11 of central tube 12. It should be understood that it may not be required to withdraw delivery tube 24 until it is removed from central tube 12 but. In another embodiment, it is only required to proximally withdraw delivery tube 24 to a point at which closure element 100 may be mounted over outer surface 11 of central tube 12 at a position distal of delivery tube 24.

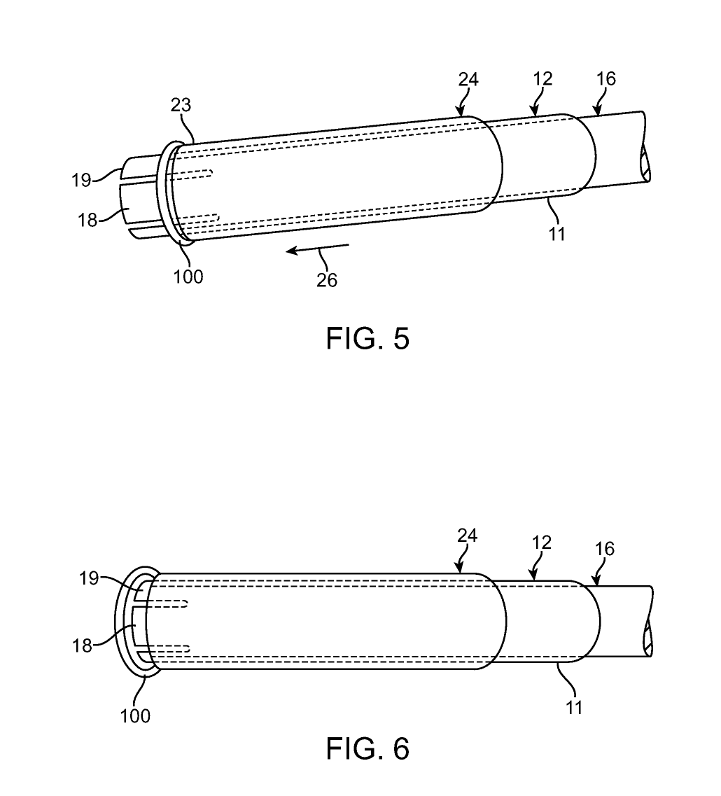

In yet another embodiment, which appears as in the embodiment of FIG. 5, but without the movement indicated by arrow 26, closure element 100 may be initially positioned, or pre-loaded, over fingers 18 distal to delivery tube 24. In embodiments wherein fingers 18 are formed with a resilient bias toward the expanded configuration, proximally withdrawing delivery tube 24 allows the splaying or flaring action of fingers 18, thereby forcing closure element 100 to slide in a proximal direction toward the bases of fingers 18. Since delivery tube 24 is not directly withdrawing closure element 100, low friction is required to permit closure element 100 to slide proximally along flared fingers 18 of central tube 12.

Once fingers 18 radially expand, their distal ends are engaged with the tissue surface so that suction, applied through suction lumens 22, will enable fingers 18 to grip the tissue surrounding arteriotomy 30. It should be understood that approximation of arteriotomy 30 in the vessel wall does not necessarily require direct engagement with the vessel wall but, instead, may be accomplished by affecting a grip on the connective tissue, such as the femoral sheath, disposed about and connected to the outer surface of the vessel.

With reference to FIG. 5, delivery tube 24 is advanced distally over central tube 12, pushing closure element 100 with distal end 23 of delivery tube 24. As delivery tube 24 passes over fingers 18, fingers 18 radially collapse inwardly, as indicated by arrows 34 in FIG. 7, into the contracted configuration. With the tissue about arteriotomy 30 gripped by fingers 18 via suction, radially collapsing fingers 18 also draws the tissue radially inward. The tissue about arteriotomy 30 becomes puckered toward a closed configuration suggested in phantom 36 at FIG. 7, thereby closing arteriotomy 30. While retaining the tissue in that configuration, guidewire 16 then may be removed. As shown in FIG. 6, closure element 100 is distally advanced by delivery tube 24 until it is pushed off of distal finger tips 19 such that closure element 100 surrounds the puckered tissue. Then, the suction is terminated so that fingers 18 release the gripped tissue, and central tube 12 and delivery tube 24 are withdrawn. Closure element 100 remains implanted around the puckered tissue in order to secure permanent closure of arteriotomy 30.

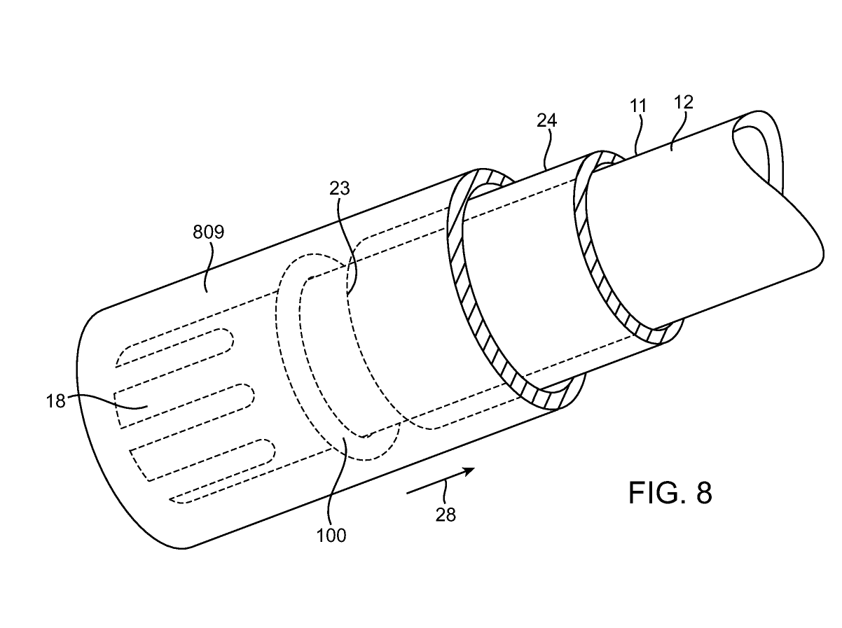

In another embodiment of the present invention illustrated in FIG. 8, a protective sheath 809 may be provided to surround delivery tube 24 as it is advanced over central tube 12 in order to facilitate tracking of closure element 100 along a tissue track to the site of the arteriotomy. In addition, protective sheath 809 may be utilized to maintain fingers 18 in the contracted position while delivery tube 24, closure element 100, and central tube 12 are tracked to the region of the arteriotomy. If such protective sheath 809 is used to maintain fingers 18 in the contracted position, it is not required to proximally withdraw delivery tube 24 to a point at which closure element 100 may be placed over outer surface 11 of central tube 12 distal of delivery tube 24 as described above. Rather, closure element 100 and delivery tube 24 are initially positioned, or pre-loaded, over outer surface 11 of central tube 12 proximal to fingers 18. Protective sheath 809 surrounds delivery tube 24, closure element 100, and central tube 12 and maintains fingers 18 in the contracted position. After the entire system reaches the region of the arteriotomy, protective sheath 809 is retracted proximally in the direction of arrow 28 in order to allow fingers 18 to deploy to the radially expanded configuration. While in the radially expanded configuration, suction is applied to cause fingers 18 to grip the adjacent tissue surface. Delivery tube 24 may then be advanced distally in order to push closure element 100 over fingers 18, and simultaneously collapsing fingers 18 radially inwardly into the contracted configuration, thus puckering the tissue about the arteriotomy. Continued advancement of delivery tube 24 results in closure element 100 being pushed off of distal finger tips 19 and around the puckered tissue.

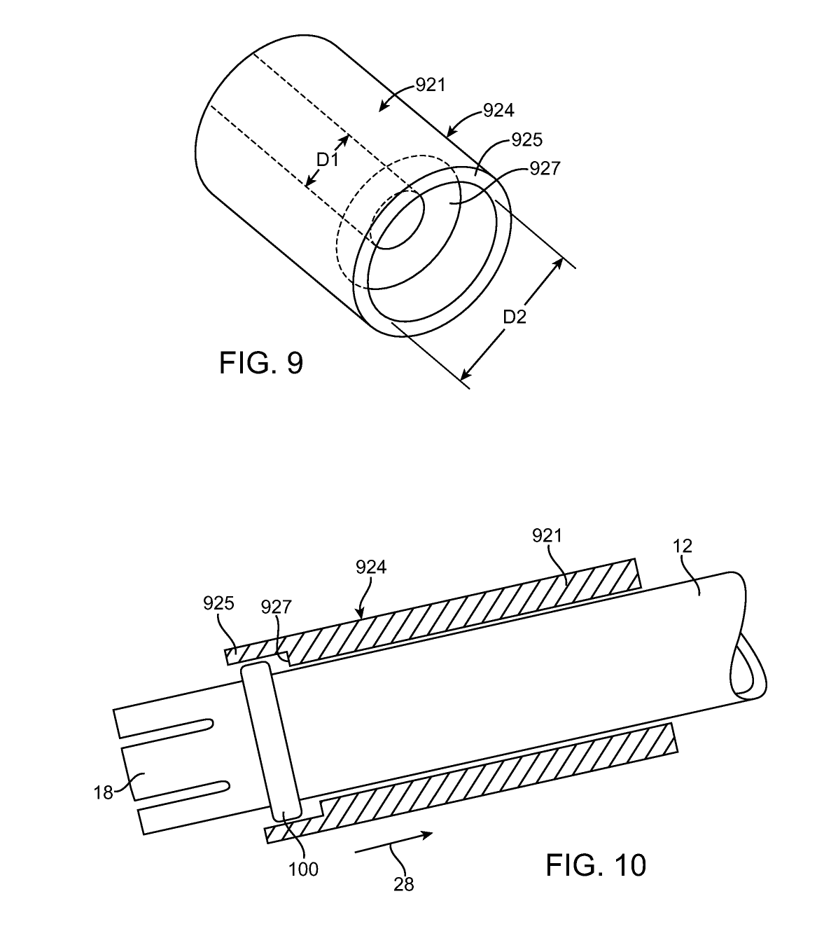

FIGS. 9 and 10 illustrate another embodiment of the delivery tube having a stepped inner diameter along the length thereof. More particularly, delivery tube 924 includes a proximal portion 921 having an inner diameter D1 and a distal portion 925 having an inner diameter D2. Inner diameter D1 of delivery tube 924 is slightly larger than outer surface 11 of central tube 12 in order to permit delivery tube 924 to slide over central tube 12. Inner diameter D2 of distal portion 925 is larger than inner diameter D1 of proximal portion 921. A step 927 is formed where the inner diameter of delivery tube 924 increases from inner diameter D1 to inner diameter D2. Delivery tube 924 is shown as having a uniform outside diameter and a corresponding change in wall thickness occurring at step 927. In an alternative embodiment (not shown), delivery tube 924 may have a change in outside diameter occurring at step 927. Step 927 is used to push the closure element over the distal tips of the fingers. As shown in FIG. 10, closure element 100 is nested within distal portion 925 of delivery tube 924 during delivery. This “nested” embodiment ensures that closure element 100 is pushed over central tube 12 with minimal deformation of the closure element, and ensures that closure element 100 is protected from contact with objects or tissue along the tissue track during advancement to the region of the arteriotomy.

In addition, distal portion 925 of delivery tube 924 maintains fingers 18 in the contracted position while delivery tube 924, closure element 100, and central tube 12 are tracked to the region of the arteriotomy. If this “nested” embodiment is used to maintain fingers 18 in the contracted position, it is not required to proximally withdraw the delivery tube to a point at which the closure element may be placed over the outer surface of the central tube distal of the delivery tube as described above. Rather, closure element 100 and delivery tube 924 are pre-loaded onto central tube 12 with distal portion 925 and closure element 100 positioned around fingers 18, thus maintaining fingers 18 in the contracted position. After the entire system reaches the region of the arteriotomy, delivery tube 924 is retracted proximally in the direction of arrow 28 in order to allow fingers 18 to deploy to the radially expanded configuration. As described above, proximally withdrawing delivery tube 924 allows the splaying or flaring action of fingers 18, thereby forcing closure element 100 to slide in a proximal direction toward the bases of fingers 18. While in the radially expanded configuration, suction is applied to cause fingers 18 to grip the adjacent tissue surface. Delivery tube 924 may then be advanced distally such that step 927 of the inner diameter of delivery tube 924 pushes closure element 100 over fingers 18, radially collapsing them inwardly into the contracted configuration and puckering the tissue, until closure element 100 is forced off of finger tips 19 and around the puckered tissue. In this embodiment, delivery tube 924 is advanced distally to a point where step 927 is distally past fingertips 19.

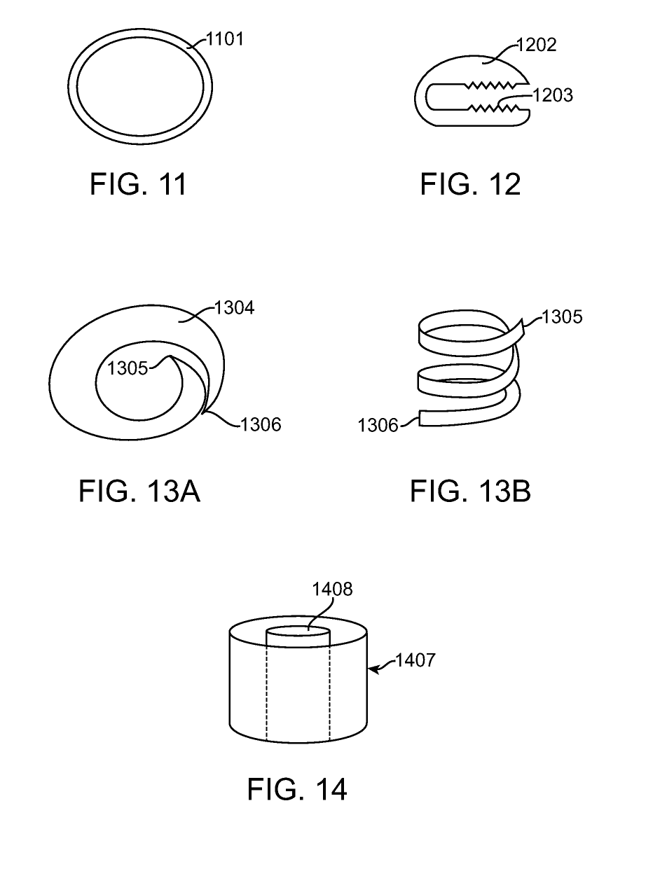

The closure element for securing permanent closure of the arteriotomy may assume various forms. For example, the closure element may be an annular band 1101 as shown in FIG. 11. In one embodiment, annular band 1101 is constructed from an elastic material that is stretched over outer surface 11 of central tube 12 so that when it is pushed off finger tips 19, annular band 1101 contracts around the puckered tissue of an arteriotomy. In other words, due to elastomeric properties, band 1101 seals and compresses tissue around the arteriotomy. Annular band 1101 may be a ring of elastomer with a rectangular, round or 0-shaped cross-section. For example, annular band 1101 may be formed from a type of biocompatible elastomer, including synthetic rubbers such as silicone or thermoplastic elastomers such as polyurethanes or polyamides. In one embodiment, the closure element may be formed from a bioabsorbable or biodegradable material that is selected to be absorbed or degraded in vivo over time. Annular band 1101 elastically grips the puckered tissue in order to secure permanent closure of the arteriotomy and thereby prevent slippage annular band 1101.

Another embodiment of the closure element is depicted in FIG. 12, wherein the closure element is a gripping clip 1202 having a generally U-shaped configuration. The inner surface of gripping clip 1202 includes teeth 1203 for gripping the puckered tissue about the arteriotomy. Alternatively, the inner surface 1202 may have other protrusions or be otherwise jagged or raised in order to grip the puckered tissue about the arteriotomy and thereby prevent slippage of gripping clip 1202.

Gripping clip 1202 may be formed of a material having resilient properties in order to grip the puckered tissue in order to secure permanent closure of the arteriotomy and further prevent slippage of gripping clip 1202. Biocompatible metals suitable for use in gripping clip 1202 include stainless steel 316L, stainless steel 316 LVM, titanium, nickel-titanium (nitinol) or bioabsorbable magnesium, which is absorbed by a patient's body as the arteriotomy into which gripping clip 1202 is inserted heals. Biocompatible non-resorbable polymeric materials suitable for use in gripping clip 1202 may include polymethylmethacrylate (PMMA), high density polyethylene (HDPE), and ultra high molecular weight polyethylene (UHMWPE). Gripping clip 1202 may also be made of an implant grade bioabsorbable polymer material such that gripping clip 1202 is absorbed by a patient's body as the arteriotomy around which gripping clip 1202 is inserted heals. For example, and not by way of limitation, gripping clip 1202 may be made from polyglycolic acid (PGA), polylactic acid (PLA), alloys or blends of PGA and PLA, alloys or blends of PGA and tri-methyl carbonate, and alloys or blends of PLA and tri-methyl carbonate. Gripping clip 1202 having sufficiently elastic properties may be stretched over the outer surface 11 of central tube 12 so that when it is released or pushed off the finger tips 19, it contracts around the puckered tissue of an arteriotomy.

Another embodiment of the closure element is depicted in FIGS. 13A and 13B, wherein the closure element is a coil spring or spiral clip 1304 having multiple loops or turns extending between a first end 1305 and a second end 1306. In one embodiment, spiral clip 1304 is constructed from an elastic material that is radially stretched over the outer surface 11 of central tube 12 so that when it is released or pushed off finger tips 19, spiral clip 1304 radially contracts around the puckered tissue of an arteriotomy. Spiral clip 1304 may be formed from any of the materials described above regarding gripping clip 1202. Spiral clip 1304 elastically grips the puckered tissue in order to secure permanent closure of the arteriotomy and thereby prevent slippage of spiral clip 1304.

Another embodiment of the closure element is depicted in FIG. 14, wherein the closure element is a plug 1407. Plug 1407 is constructed out of a hydrogel, collagen, or a bioabsorbable polymer having swelling potential such that when it is released or pushed off finger tips 19, plug 1407 swells around the puckered tissue and plugs the tissue track adjacent the arteriotomy, thereby securing permanent closure of the arteriotomy. In one embodiment, the hydrogel, collagen, or bioabsorbable polymer material may be freeze-dried or dehydrated. Plug 1407 includes a central passageway or hole 1408 extending there through so that plug 1407 may be delivered over outer surface 11 of central tube 12. Central passageway or hole 1408 will swell shut after plug 1407 is released from central tube 12.

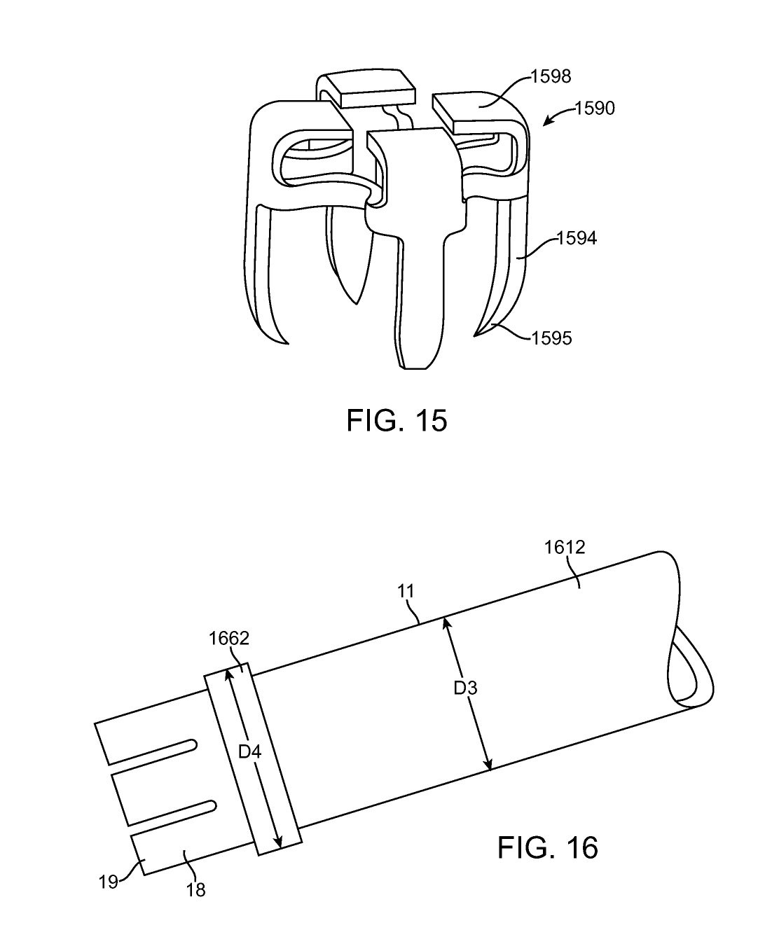

Another embodiment of the closure element is depicted in FIG. 15, wherein the closure element is a staple 1590. Staple 1590 may be similar to one of the staples, for example, of the type described in U.S. Pat. No. 6,767,356 (Kanner). Reference is made to the Kanner '356 patent for additional details concerning various constructions and embodiments of the staple closure element, which are incorporated by reference herein, in their entirety.

Staple 1590 is delivered over a central tube 1612, shown in FIG. 16. Central tube 1612 includes at least one ridge or protrusion 1662 about outer surface 11 proximal to fingers 18. Ridge 1662 closes prongs 1594 of staple 1590 about the arteriotomy. Ridge 1662 is provided circumferentially about outer surface 11 and may be continuous or non-continuous. Ridge 1662 has a greater outer diameter D4 than diameter D3 of outer surface 11 of central tube 1612 for engaging and subsequently closing staple 1590.

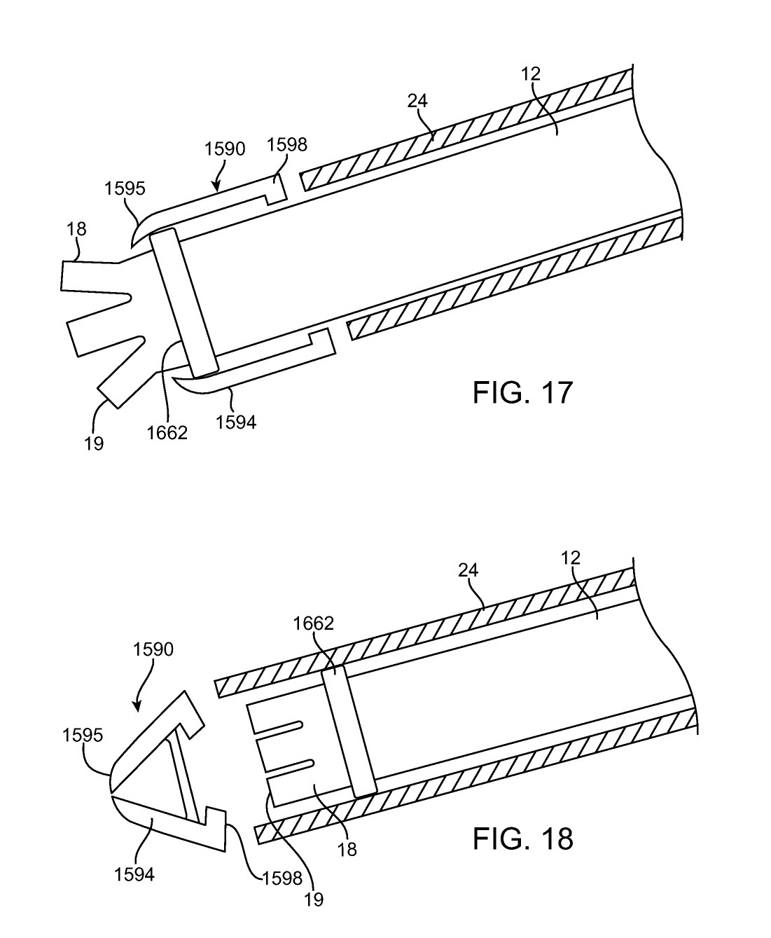

FIGS. 17 and 18 illustrate delivery of staple 1590 using central tube 1612 and delivery tube 24. Staple 1590 is mounted about central tube 1612 with prongs 1594 pointing distally and extending across ridge 1662, as shown in FIG. 17. Fingers 18 are splayed for engagement with tissue, as described regarding other embodiments herein. As delivery tube 24 is advanced to push staple 1590 distally along central tube 1612, prongs 1594 contract fingers 18 and pucker tissue surrounding the arteriotomy. Staple tips 1595 are slid over finger tips 19 to pierce the puckered tissue. Further advancement of staple 1590 by delivery tube 24 forces staple base portions 1598 to radially expand over ridge 1662, thus causing staple prongs 1594 to pivot such that staple tips 1595 contract radially inwards towards each other, as shown in FIG. 18. Staple 1590 remains plastically deformed and embedded around the puckered tissue in order to secure permanent closure of the arteriotomy.

As previously stated, fingers 18 may be moved between a radially contracted, low profile configuration (FIGS. 1, 2, 5, and 6) and a radially expanded, deployed configuration (FIGS. 3 and 4) by delivery tube 24 that is slidably disposed about central tube 12. Fingers 18 may move in a generally radial pattern with or without symmetry about a central point, such as a central location of the arteriotomy. Alternatively, some or all of fingers 18 may be adapted to expand and contract in a pattern having line symmetry (not shown). Thus, the terms “radial” or “radially” are not intended herein to be limited strictly to the radii of a circle when such terms are used to describe the movement of fingers 18.

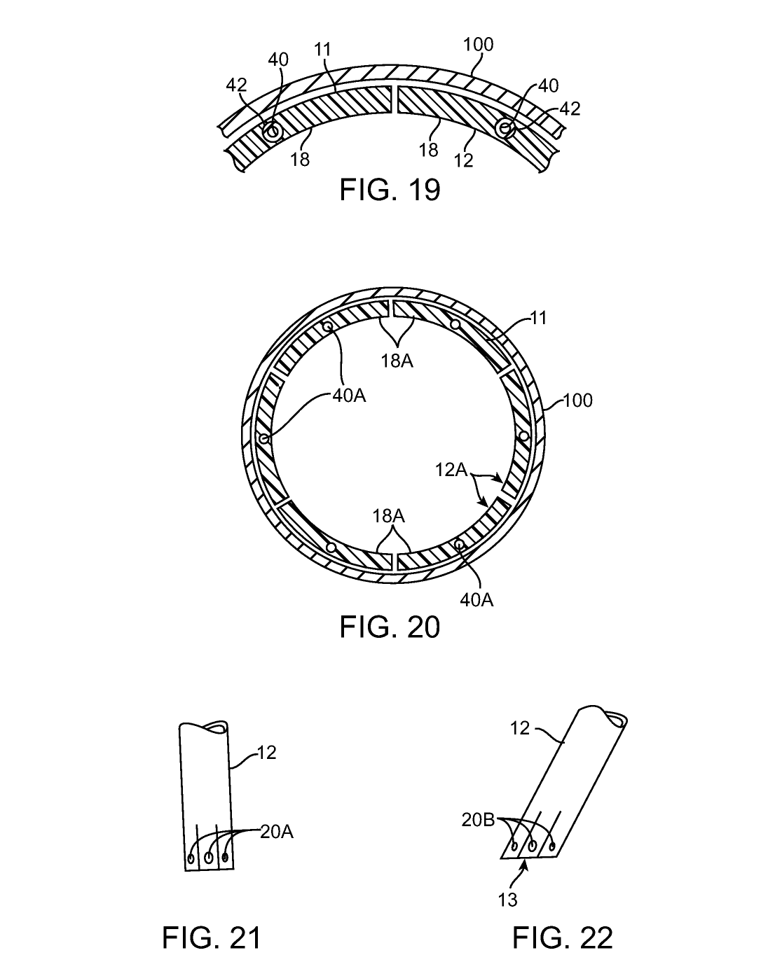

FIG. 19 illustrates one mode of construction for causing fingers 18 to deploy to a radially expanded configuration in which fingers 18 are formed with a resilient bias toward the expanded configuration. More particularly, central tube 12 may be a suitably flexible, extruded biocompatible plastic having suction lumens 40 extending through the wall of tube 12. Fingers 18 may be formed integrally with tube 12. Smaller suction tubes 42, formed from a material having sufficient elasticity and resilience may be contained within lumens 40 to communicate the suction to suction ports 20 and to assist in biasing fingers 18 in their radially outward, splayed configuration. Suction tubes 42 may, for example, be formed from nitinol (NiTi) hypotubes that may extend within the extruded lumens 40 in the wall of central tube 12. The nitinol hypotubes may be heat set to a preformed shape (bent radially outward) so that they will bias fingers 18 to the outward position of FIGS. 3 and 4. It should be understood that other resilient spring elements may be incorporated into or associated with fingers 18 to bias them toward a radially outward configuration. Fingers 18 may be contracted from the splayed to the contracted configuration by advancing delivery tube 24 distally, sliding it along outer surface 11 of central tube 12 as described in more detail above. As delivery tube 24 is advanced distally, it overcomes the bias of the nitinol hypotubes so that continued advancement of delivery tube 24 draws fingers 18 together. The nitinol hypotubes should enhance the ability for suction to be maintained at the suction outlets throughout such range of movement.

FIG. 20 illustrates another embodiment for causing fingers 18 to deploy to a radially expanded configuration in which central tube 12A is formed entirely from a shape memory alloy such as nitinol with fingers 18A being heat set to be biased in the radially expanded, splayed configuration when at normal human body temperature. Suction lumens 40A may be formed longitudinally through the wall of the tube by wire electrical discharge machining (EDM). For example, using a 0.031 inch EDM electrode would enable fabrication of a finished hole of the order of 0.035 inch in the tube wall with a depth of up to about six inches. Should it be necessary to make the device longer, additional tubes could be fabricated and joined end-to-end with suction lumens 40A aligned. The tube may, for example, be of the order of about 0.23 inch diameter with a wall thickness of about 0.072 inch and an inner diameter of about 0.09 inch. The distal portion of nitinol central tube 12A may be formed similarly to the above-described embodiments such that the plurality of fingers 18A each include suction ports at or about the distal ends of fingers 18A. The nitinol should be treated so that fingers 18A are biased to the radially expanded configuration yet are elastically returnable to the non-expanded configuration by advancement of delivery tube 24.

As will be apparent to those skilled in the art, suction ports 20 of fingers 18 may be located in various configurations. FIGS. 2, 3, and 7 show suction ports 20 located on the distal end faces of fingers 18. It may be desirable, in some cases, to locate the suction ports at other locations for contacting tissue surrounding the arteriotomy. For example, in one embodiment of the present invention shown in FIG. 21, suction ports 20A may be located on the outer faces of fingers 18. In another embodiment of the present invention shown in FIG. 22, the distal end of central tube 12 is beveled at bevel 13. The angle of bevel 13 may be selected to correspond to the approach angle (approximately 45°) of the device to the vessel. Suction ports 20B also may be aligned along bevel 13.

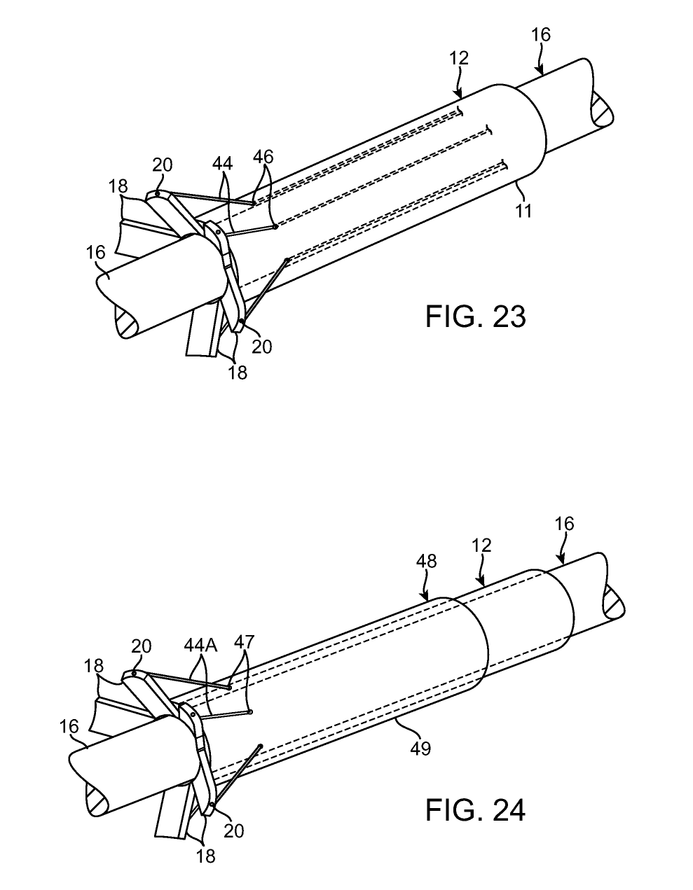

FIG. 23 illustrates an embodiment of the invention including an arrangement of pull wires for deploying fingers 18. In this embodiment, fingers 18 are radially expanded by pull wires such as filaments 44 secured to the outer ends of the fingers 18. In one embodiment shown in FIG. 23, filaments 44 may extend through apertures 46 located in central tube 12 and may extend proximally to a control point at the proximal end of the system. Apertures 46 should be located as to not interfere with suction lumens 22 extending through the wall of central tube 12. During delivery, fingers 18 should be in the contracted, low profile configuration, with or without the assistance of delivery tube 24. Once the device is located in the region of the arteriotomy, pulling on filaments 44 cause fingers 18 to pivot or flex about their roots. After suction is applied such that fingers 18 grip the tissue surrounding the arteriotomy, fingers 18 may be returned to a contracted configuration by releasing tension on filaments 44 and advancing delivery tube 24 and closure element 100 in a distal direction over outer surface 11 of central tube 12 as described above with respect to FIGS. 4-6. Closure element 100 is pushed or forced over filaments 44 and fingers 18 and is pushed off of distal tips 19 in order to permanently close the arteriotomy as described above. Filaments 44 may be of a fine diameter of the order of about 0.0015 to about 0.005 inch, and may be formed from polymers such as polypropylene, polyethylene terephthalate or nylon or from metals such as nitinol, nickel-cobalt-chromium-molybdenum superalloy, stainless steel or the like. Filaments 44 may be attached to fingers 18 by knotting, laser welding or other appropriate means for attachment as will be familiar to those skilled in the art.

FIG. 24 illustrates another embodiment of the invention including an arrangement of pull wires for deploying fingers 18. In the embodiment of FIG. 24, filaments 44A may be secured directly to a pull wire sheath 48 at attachment points 47. Pull wire sheath 48 is slidably located over outer surface 11 of central tube 12 and may extend proximally to a control point at the proximal end of the system. During delivery, fingers 18 should be in the contracted, low profile configuration by distally advancing pull wire sheath 48 over fingers 18. Once the device is located in the region of the arteriotomy, retraction of pull wire sheath 48 will tension filaments 44A to expand fingers 18 as shown in FIG. 24. After suction is applied such that fingers 18 grip the tissue surrounding the arteriotomy, fingers 18 may be returned to a contracted configuration by distally advancing pull wire sheath 48. Distally advancing pull wire sheath 48 pushes or forces fingers 18 into the contracted configuration. In another embodiment in which fingers 18 are formed resiliently biased to the contracted configuration, distally advancing pull wire sheath 48 merely releases tension in filaments 44A to allow fingers 18 to close. Alternatively (not shown), fingers 18 may be returned to a contracted configuration by distally advancing delivery tube 24 and closure element 100 over outer surface 49 of pull wire sheath 48 and over filaments 44A. Regardless of how fingers 18 are closed (via distal advancement of pull wire sheath 48 or delivery tube 24), delivery tube 24 and closure element 100 are then advanced in a distal direction over an outer surface 49 of pull wire sheath 48 in order to push or force closure element 100 off the distal end of the device to permanently close the arteriotomy. Filaments 44A may be of a fine diameter of the order of about 0.0015 to about 0.005 inch, and may be formed from polymers such as polypropylene, polyethylene terephthalate or nylon or from metals such as nitinol, nickel-cobalt-chromium-molybdenum superalloy, stainless steel or the like. In addition, filaments 44A may be attached to fingers 18 and pull wire sheath 48 by knotting, laser welding or other appropriate means for attachment as will be familiar to those skilled in the art.

If an arrangement of pull wires such as those described above with reference to FIGS. 23-24 is used to maintain fingers 18 in the contracted position, it is not required to proximally withdraw delivery tube 24 to a point at which closure element 100 may be placed over outer surface 11 of central tube 12 distal of delivery tube 24. Since retraction of delivery tube 24 is not required for causing deployment of fingers 18, closure element 100 and delivery tube 24 may initially be positioned, or pre-loaded, over outer surface 11 of central tube 12 proximal to fingers 18. After the pull wire arrangement is utilized for deploying fingers 18 to the radially expanded configuration, delivery tube 24 and closure element 100 are then advanced in a distal direction in order to push or force closure element 100 off the distal end of the device to permanently close the arteriotomy.

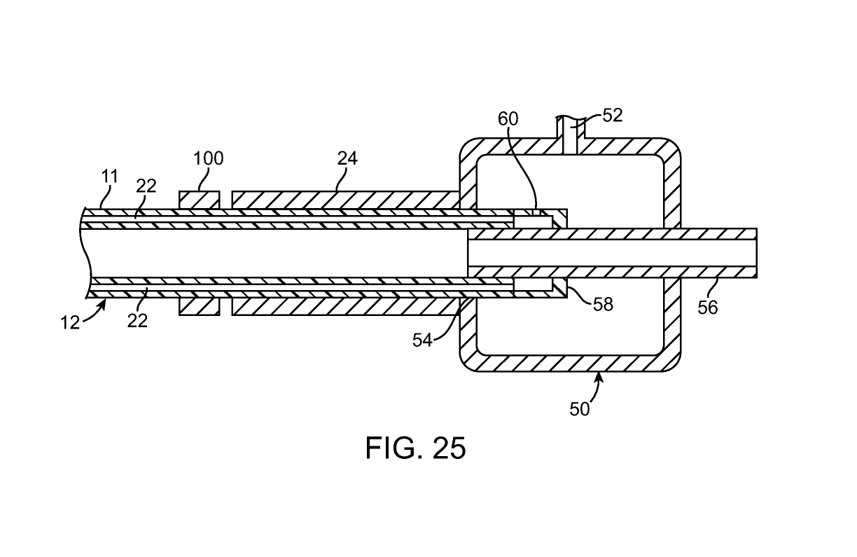

FIG. 25 illustrates one arrangement at the proximal end of the device by which the device may be controlled. The proximal end of the device may include a sealed housing 50 secured to the proximal end of delivery tube 24. Housing 50 includes a suction port 52 that is connectible to a source of suction (not shown). Central tube 12 extends through a slidably sealed opening 54 in housing 50 and includes a proximally extending extension tube 56 adapted to receive a guidewire. The device also includes a manifold 58 that is secured to the proximal end of central tube 12. Manifold 58 is in fluid communication with the proximal ends of suction lumens 22 in central tube 12. When located within housing 50, manifold 58 also is in fluid communication with the interior of housing 50 by one or more ports 60 so that when suction is applied to allow fingers 18 to grip the tissue surrounding the arteriotomy, the suction will be communicated from housing 50 to distal suction ports 20 through suction lumens 22. Housing 50 and delivery tube 24 thus are movable together with respect to central tube 12 and manifold 58 in order to deliver closure element 100 over the distal end of the device, and also in order to expand and/or collapse the fingers at the distal end of the device.

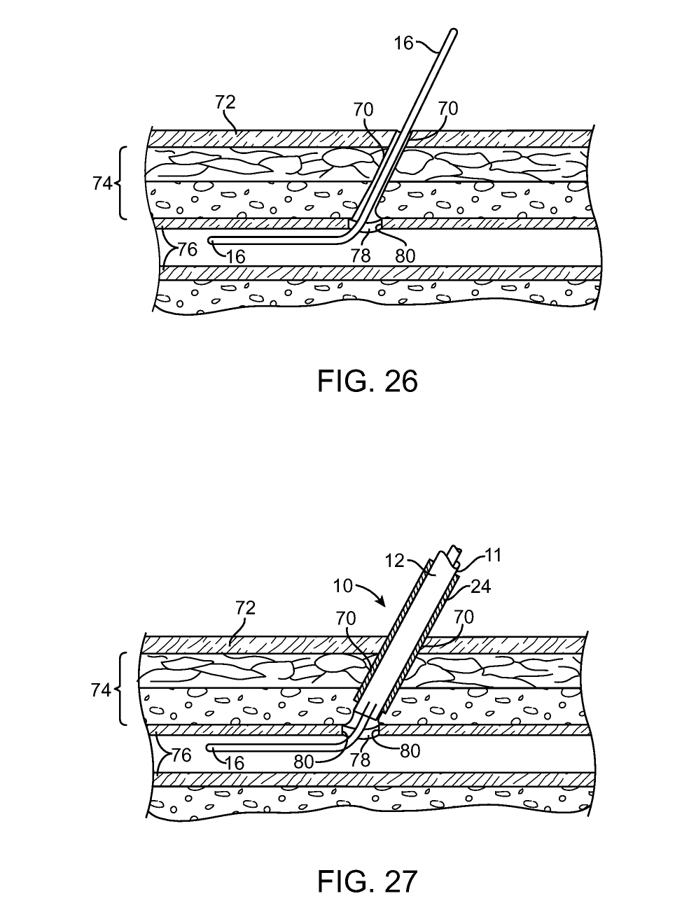

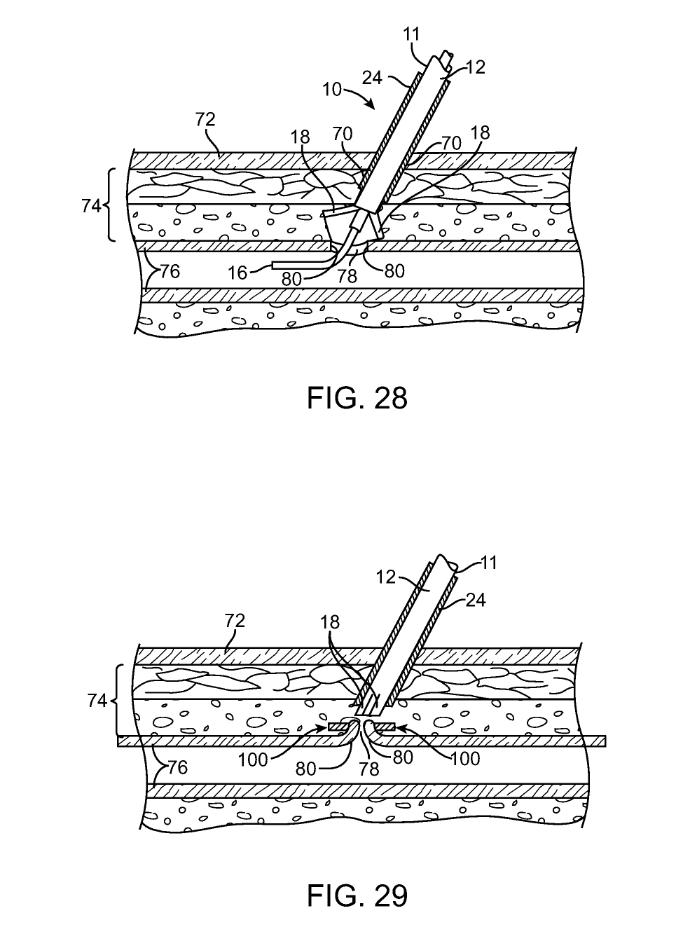

FIGS. 26-29 illustrate, diagrammatically, the manner in which the device may cause approximation of the edges of the arteriotomy without directly engaging the vessel wall. As shown on FIG. 26, after the vascular procedure has been completed, guidewire 16 is placed (or left in place) through a tissue track 70 and into a vessel 76 and the introducer (not shown) then is withdrawn. Tissue track 70 extends through skin 72, subcutaneous and connective tissue 74, including fascia and the femoral sheath which are attached to the outer adventitia of vessel 76. FIG. 27 illustrates the distal portion of the device advanced along indwelling guidewire 16 through tissue track 70 as it approaches connective tissue 74 disposed about the region of an arteriotomy 78. Delivery tube 24 is positioned over outer surface 11 of central tube 12 in order to maintain fingers 18 in the contracted or collapsed configuration. When the device is positioned proximally of an arteriotomy 78, delivery tube 24 is retracted to enable or cause the fingers to be deployed radially outward into engagement with connective tissue 74 about and proximally of the puncture in the vessel wall as shown in FIG. 28. Suction then is applied to cause connective tissue 74 to be drawn securely against the suction ports. The connection between connective tissue 74 and the wall of vessel 76 is such that connective tissue 74 can be drawn while maintaining its attachment to the vessel wall.

As previously explained with relation to FIGS. 3-4, it may be required to proximally withdraw delivery tube 24 until the distal end thereof is released from central tube 12 and/or guidewire 16. If so required, delivery tube 24 is removed and closure element 100 is positioned over the proximal end of outer surface 11 of central tube 12. In addition, delivery tube 24 is also positioned over the proximal end of outer surface 11 of central tube 12, proximal to closure element 100. Delivery tube 24 is advanced distally in order to push or force closure element 100 over outer surface 11 of central tube 12. As delivery tube 24 and closure element 100 are advanced over fingers 18, fingers 18 radially collapse inwardly into the contracted configuration such that the connective tissue 74 gripped by fingers 18 will also be drawn together and that, in turn, draws edges 80 of arteriotomy 78 toward each other by reason of the connection between connective tissue 74 and the wall of vessel 76 (see FIG. 29). With connective tissue 74 so held by the device, guidewire 16 may be withdrawn and closure element 100 is distally advanced by delivery tube 24 until it is pushed or forced over or past the distal tips of fingers 18 such that closure element 100 engages the puckered connective tissue 74 as illustrated in FIG. 29. The suction or aspiration force is terminated so that fingers 18 release connective tissue 74, and central tube 12 and delivery tube 24 are withdrawn. Closure element 100 remains around the puckered connective tissue 74 in order to secure permanent closure of arteriotomy 78. After removal of the delivery device, a short duration of external pressure may be desirable as a precaution.

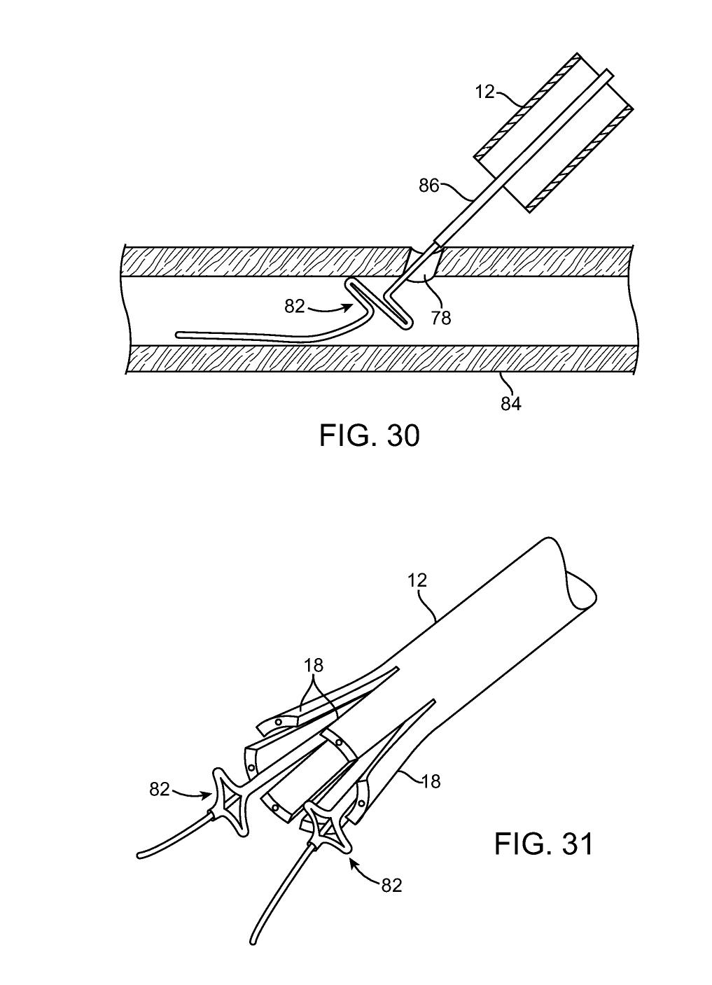

The invention also may be practiced in conjunction with a stabilizing device, for example, of the type described in U.S. Pat. No. 6,767,356 (Kanner). As shown in FIG. 30, wire-like stabilizers 82 may extend through central tube 12. The distal ends of stabilizers 82 are configured to be placed through arteriotomy 78 into the lumen of vessel 84. Stabilizers 82, which are inserted in a linear configuration, then are operated to an enlarged configuration, as illustrated in FIGS. 30 or 31, so that they cannot be withdrawn through arteriotomy 78. Stabilizers 82 thus provide a stable platform with which central tube 12 can be held in centered position over the region of the arteriotomy. Stabilizers 82 may include a guide 86 that may be secured to tube 12 and through which stabilizer 82 may be advanced or retracted when in its linear, non-deployed configuration. Reference is made to the Kanner '356 patent for additional details concerning various constructions and embodiments of the stabilizing system, which are incorporated by reference herein, in their entirety.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A system for closing an arteriotomy comprising:
an elongate central tube having a distal portion including a plurality of gripping elements disposed thereon, the plurality of gripping elements configured to be movable between a radially contracted configuration and a radially expanded configuration, wherein the gripping elements are adapted to effect a grip on tissue surrounding the arteriotomy when the gripping elements are in the radially expanded configuration;
a delivery tube disposed about the central tube, the delivery tube being slidable along the central tube to urge the gripping elements from the radially expanded configuration to the radially contracted configuration while maintaining grip on the tissue whereby the tissue may be puckered together; and
a closure element disposed on an outside surface of the central tube, the closure element being slidably advanceable along and off a distal end of the central tube by urging of the delivery tube and adapted to surround the puckered tissue to close the arteriotomy.

2. The system of claim 1, wherein the gripping elements comprise suction ports.

3. The system of claim 2, further comprising a plurality of suction lumens extending longitudinally through the wall of the central tube, each of the suction lumens being in communication with a corresponding suction port.

4. The system of claim 1, wherein the distal portion of the central tube comprises a plurality of fingers integral with the central tube, the fingers being movable relative to a body of the central tube for locating the gripping elements radially spaced from the body of the central tube in the radially expanded configuration.

5. The system of claim 4, wherein the fingers are resiliently biased radially outwardly and the delivery tube is engageable with the fingers to thereby urge the gripping elements from the radially expanded configuration to the radially contracted configuration.

6. The system of claim 4, further comprising a pull wire associated with each of the fingers, the pull wires being arranged to selectively draw the gripping elements from the radially contracted configuration to the radially expanded configuration.

7. The system of claim 1, wherein the closure element is an annular band of material having elastic properties to grip the tissue surrounding the arteriotomy.

8. The system of claim 1, wherein the closure element is a U-shaped gripping clip including protrusions on the inner surface thereof for engaging the tissue surrounding the arteriotomy.

9. The system of claim 1, wherein the closure element is a spiral band having a plurality of turns, the spiral band formed of a material having elastic properties to grip the tissue surrounding the arteriotomy.

10. The system of claim 1, wherein the closure element is a staple having at least two prongs for embedment in the tissue surrounding the arteriotomy.

11. The system of claim 1, wherein the closure element is a plug having a central passageway therethrough, the plug formed of a material having swelling potential to effectively plug the arteriotomy.

12. The system of claim 1, wherein the delivery tube has a uniform inner diameter along the length thereof and a distal end of the delivery tube slidably advances the closure element.

13. The system of claim 1, wherein a distal portion of the delivery tube has a larger inner diameter than a proximal portion of the delivery tube and an internal step formed between the distal portion and the proximal portion slidably advances the closure element.

14. A method for closing an arteriotomy comprising the steps of:
providing a plurality of tissue gripping elements on a distal portion of a central tube, the gripping elements being configured to be selectively movable between a radially contracted configuration and a radially expanded configuration;
positioning the gripping elements in surrounding relation to the arteriotomy and deploying them in the radially expanded configuration;
actuating the gripping elements to cause them to grip a tissue surface without piercing the tissue;
providing a closure element over an outside surface of the central tube;
providing a delivery tube slidably disposed around the central tube such that the delivery tube can push the closure element along the outside surface of the central tube;
advancing the delivery tube and thereby the closure element distally along the outside surface of the central tube until the gripping elements are collapsed to the radially contracted configuration while maintaining grip on the tissue surface whereby the tissue surrounding the arteriotomy may be puckered together;
further slidably advancing the delivery tube and thereby the closure element distally along the outside of the central tube until the closure element is pushed off of a distal end of the central tube by the delivery tube such that the closure element surrounds the puckered tissue and secures the arteriotomy closed; and
after the closure element secures the arteriotomy closed, releasing the gripping elements from the tissue.

15. The method of claim 14, wherein the gripping elements comprise suction ports and the step of actuating the gripping elements comprises applying suction to the suction ports.

16. The method of claim 14, wherein the closure element is an annular band of material having elastic properties to grip the tissue surrounding the arteriotomy.

17. The method of claim 14, wherein the closure element is a U-shaped gripping clip including protrusions on the inner surface thereof for engaging the tissue surrounding the arteriotomy.

18. The method of claim 14, wherein the closure element is a spiral band having a plurality of turns, wherein the spiral band is formed of a material having elastic properties to grip the tissue surrounding the arteriotomy.

19. The method of claim 14, wherein the closure element is a staple having at least two prongs for embedment in the tissue surrounding the arteriotomy.

20. The method of claim 14, wherein the closure element is a plug having a central passageway therethrough, the plug formed of a material having swelling potential to effectively plug the arteriotomy.

21. The method of claim 14, wherein the delivery tube has a uniform inner diameter along the length thereof and a distal end of the delivery tube slidably advances the closure element.

22. The method of claim 14, wherein a distal portion of the delivery tube has a larger inner diameter than a proximal portion of the delivery tube and an inner step formed between the distal portion and the proximal portion slidably advances the closure element.
